(19) United States
(12) Patent Application Publication
(10) Pub. No.: US 2017/0228826 A1
(43) Pub. Date: Aug. 10, 2017

(54) IDENTIFYING MARKED ARTICLES IN THE INTERNATIONAL STREAM OF COMMERCE

(71) Applicant: APDN (B.V.I.) Inc., Tortola (VG)

(72) Inventors: James A. Hayward, Stony Brook, NY (US); MeiLin Wan, Brightwaters, NY (US)

(73) Assignee: APDN (B.V.I.) Inc., Tortola (VG)

(21) Appl. No.: 15/427,983

(22) Filed: Feb. 8, 2017

Related U.S. Application Data
(60) Provisional application No. 62/292,544, filed on Feb. 8, 2016.

(51) Int. Cl. G06Q 40/00; G06K 19/06; C12Q 1/68

(52) U.S. Cl. CPC G06Q 40/10; C12Q 1/6816; G06K 19/0614

(57) ABSTRACT

A method and system of identifying articles in commerce including tagging a raw material during said raw material's manufacturing process with a unique molecular marker in a home country in which the raw material was produced. The marker identifies home country information. The tagged raw material is transported from the home country to a foreign country. The tagged raw material is processed in the foreign country to generate a processed product. The processed product is transported to the home country. The processed product is analyzing to determine the presence of the marker in the processed product. A determination is made whether the raw material originated in the home country in response to the detection of the marker.

This application claims the benefit of priority to U.S. Provisional Patent Application Ser. No. 62/292,544 filed on Feb. 8, 2016, the contents of which are incorporated by reference herein.

### FIELD OF THE INVENTION

The present invention relates to a method for identifying marked articles in the international stream of commerce. More particularly, exemplary embodiments of the present invention relate to identifying marked articles included in value-added products returning to a home country from the international chain of commerce. Exemplary embodiments of the present invention relate to using nucleic acid taggants to uniquely identify marked articles.

### BACKGROUND

Billions of dollars of raw materials are harvested in the United States annually. Generally, raw materials are distributed through the stream of commerce and used to assemble products including the raw material. For example, raw cotton is harvested, ginned, and distributed through the stream of commerce and used to manufacture textiles and apparel.

In some instances, raw or partially processed materials are exported from the United States, thus entering the international chain of commerce. The raw or partially processed materials may be assembled into partially or fully formed products in one or more countries outside the United States and then shipped back into the United States for further assembly, processing or sale. The partially or fully formed products shipped back to the United States may be referred to as value-added products. The value-added products may include the raw material originally harvested in the United States, as well as additional materials. Thus, it may be desirable to be able to establish the authenticity of the raw materials included in the value-added products. For example, cotton harvested in the United States may vary in quality, and it may be desirable to determine the presence and quantity of a particular species or cultivar of cotton included in the value-added product. Further, it may be desirable to authenticate the provenance of cotton or other raw material originating somewhere in the United States and included in a value-added product re-entering the United States. Authentication may likewise be desirable to authenticate the providence of any raw material originating from any home country and included in a value-added product re-entering the home country.

### SUMMARY

The present invention provides a method of identifying articles in commerce including tagging a raw material during said raw material's manufacturing process with a unique molecular marker in a home country in which the raw material was produced. The marker identifies home country information. The tagged raw material is transported from the home country to a foreign country. The tagged raw material is processed in the foreign country to generate a processed product. The processed product is transported to the home country. The processed product is analyzing to determine the presence of the marker in the processed product. A determination is made whether the raw material originated in the home country in response to the detection of the marker.

The present invention also provides a method of tracking articles in international commerce including transporting a raw material tagged with a nucleic acid marker from a home country in which the raw material was produced to a non-home country, wherein said marker is associated with home country information. The raw material is subjected to a processes in the non-home country to generate a value added product. The value added product is transported to the home country. The value added product is subjected to an interrogation by an in-field detection device to detect the presence or absence of the nucleic acid marker. In response to the nucleic acid marker being detected, a first tariff and/or tax condition is determined and in response to the nucleic acid marker not being detected a second tariff and/or tax condition is determined.

The present invention further provides a system of tracking articles in international commerce including a taggent including a molecular marker applied to raw material. The taggent includes information associated with a country of origin. A detection device is provided for detecting the taggent in processed goods containing the raw material after the raw material has been processed to obtain country or origin information. An analysis device operably connected to the detection device is provided for analyzing the detected taggent.

### BRIEF DESCRIPTION OF THE FIGURES

The above and other features of the present invention will become more apparent by describing in detail exemplary embodiments thereof, with reference to the accompanying drawings, in which:

FIG. 1 illustrates a schematic diagram showing a raw material moving through the international stream of commerce according to an exemplary embodiment of the present invention.

FIG. 2 illustrates a schematic diagram showing a product moving through the international stream of commerce according to an exemplary embodiment of the present invention.

FIG. 3 illustrates an exemplary method of authenticating a taggant of a marked raw material or a marked product included in a value-added product according to an exemplary embodiment of the present invention.

FIG. 4 illustrates an embodiment of a process according to the present invention

FIG. 5 illustrates an exemplary in-field detection instrument according to an exemplary embodiment of the present invention.

### DETAILED DESCRIPTION

Exemplary embodiments of the present invention may provide a method of authenticating tagged materials returning to a home country in a value-added product from the international stream of commerce.

Exemplary embodiments of the present invention may provide a method of authenticating tagged cotton harvested in a home country (e.g., the United States) and returning to the home country as part of a value-added product that is fully or partially assembled in one or more foreign countries. The value-added product may pass through a customs inspection location in the home country.

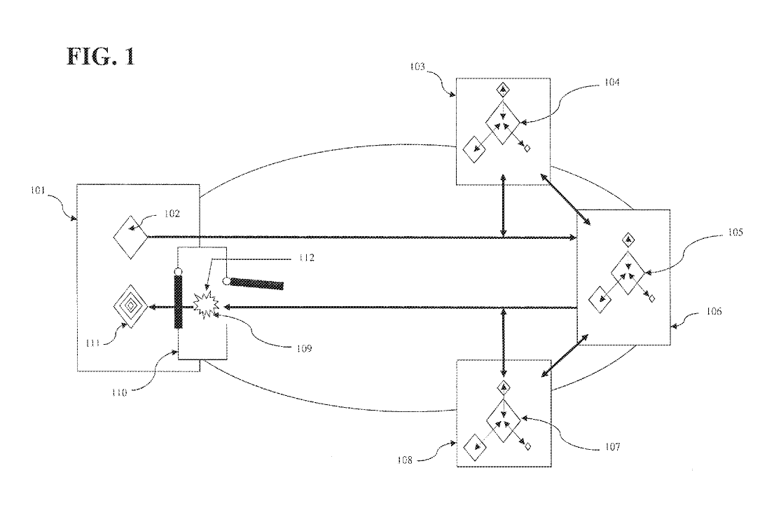

FIG. 1 illustrates a schematic diagram showing a raw material moving through the international stream of commerce according to an exemplary embodiment of the present invention.

Referring to FIG. 1, a raw material may be tagged in a home country 101 to form a tagged raw material 102. The tagged raw material 102 may be tagged by at least one of a number of methods of tagging the raw material according to exemplary embodiments of the present invention. Methods of tagging a raw material according to an exemplary embodiment of the present invention are described below in more detail.

According to an exemplary embodiment of the present invention, the raw material may include a textile, a fiber, cotton, ginned cotton, a cotton blend, wool, yarn, nylon, cashmere, a synthetic fabric and a synthetic fabric blend. The raw material may include one or more raw fibers, such as one or more raw cotton fibers. The raw material may include a textile material including yarns, threads, fabrics, nonwoven materials, and products manufactured from fibrous materials. The raw materials may also include various electronic components such as microchips.

The tagged raw materials 102 may be introduced to the international stream of commerce, and may be delivered to one or more foreign countries (e.g., a first foreign country 103, a second foreign country 106 and/or a third foreign country 108) for processing to generate a value added product 111.

According to an exemplary embodiment of the present invention, the tagged raw materials 102 may be sent directly to a single foreign country (e.g., the first foreign country 103) and then returned to the home country 101 after the processing/value-added steps are performed. Alternatively, the tagged raw materials 102 may be sent to the first foreign country 103 for processing and then sent to one or more additional foreign countries (e.g., the second foreign country 106 and/or the third foreign country 108) for additional processing and to generate the value-added product 111. For example, the tagged raw materials 102 may undergo a first processing/value-added step 104 in the first foreign country 103, a second processing/value-added step 105 in the second foreign country 106, and/or a third processing/value-added step 107 in the third foreign country 108 prior to being returned to the home country 101. That is, the value added product 111 may include the tagged raw materials 102 originating in the home country 101.

Processing or value-added steps (e.g., 104, 105 and/or 107) may include generating a fully or partially completed product. For example, when the tagged raw materials 102 include cotton, the cotton may be shipped out of a home country and spun and/or woven into sheets, and assembled into substantially complete articles or apparel in one or more foreign countries. The cotton may be blended with one or more other materials, as desired; to generate textile or apparel articles including cotton blends. Thus, a partially or fully assembled textile or apparel article may arrive at the home country including the raw cotton originating in the home country.

Prior to, or upon, entering the home country 101, the value added product 111 may pass through a customs inspection location 110. For example, a value-added product 111 entering the United States may pass through a U.S. Customs and Border Protection location prior to entering United States commerce. One role of the customs inspection location 110 may be to perform a tariff determination 109. The term “tariff” as used herein shall mean any tax or duty levied upon a value-added product upon importation into a home country. According to an exemplary embodiment of the present invention, one or more taggants or markers (e.g., a nucleic acid marker) included in the marked raw materials may be authenticated 112 at the customs inspection location 110. That is, the goods may be interrogated to determine the presence or absence of the nucleic acid marker in the value-added product 111. For example, the authentication may occur prior to or at the time of assessing any tariff(s) that may be due on products entering the country. That is, tariff(s) may be due on foreign derived raw materials or foreign derived products, but not on products or raw materials that originate in the home country. Thus, it may be determined that some portion or some percentage of the value-added product 111 is not subject to one or more tariff(s) because the value-added product 111 includes one or more raw materials which originated in the home country 101.

According to an exemplary embodiment of the present invention, the one or more tariff(s) may not be due on a percentage by weight of the product. For example, if the raw material includes cotton, a tariff may only be due on the increased weight added to the raw material during processing the raw material in the one or more foreign countries. Alternatively, the tariff(s) may only be due on the increase in value from raw cotton to a completed textile article.

The authenticated value-added product 111 including the tagged raw materials 102 may then pass into the home country 101 after passing through the customs inspection location 110.

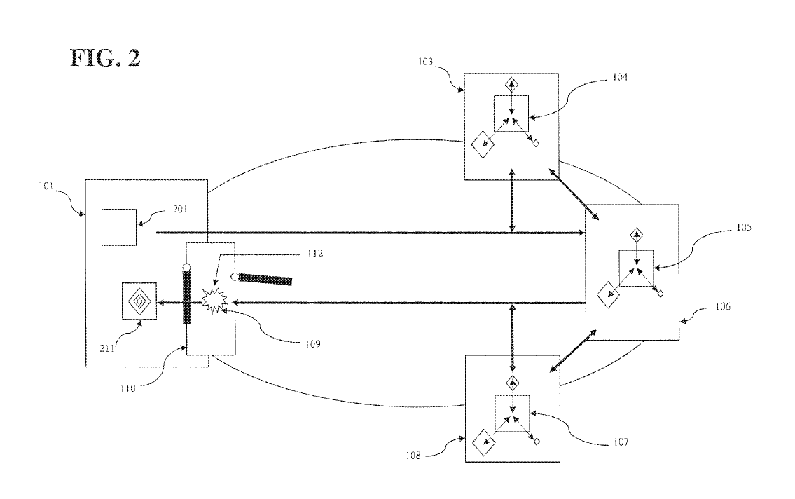

FIG. 2 illustrates a schematic diagram showing a product moving through the international stream of commerce according to an exemplary embodiment of the present invention.

Referring to FIG. 2, a product may be tagged in the home country 101 to form a marked product 201. The marked product 201 may be tagged by at least one of a number of methods of tagging the product according to exemplary embodiments of the present invention. Methods of tagging a product according to an exemplary embodiment of the present invention are described below in more detail.

According to an exemplary embodiment of the present invention, the product may include an electronics product, which may be marked to generate the marked product 201. The electronics product may include a computer, a computer component, a network component, a computer disk, a microchip, a microcircuit, a semiconductor, a diode, a transistor, an integrated circuit, an optoelectronic device, a digital display, a vacuum tube, a discharge device, a power source, a resistor, a capacitor, a battery, a magnetic device, a sensor, a detector, a transducer, an electronics assembly, a terminal, a cable, and a switch.

According to an exemplary embodiment of the present invention, the product may include a liquid, which may be marked to generate the marked product 201. The liquid may include an ink, a solvent, an alcohol, and an adhesive.

According to an exemplary embodiment of the present invention, the product may include a commodity, which may be marked to generate the marked product 201. The commodity may include iron ore, crude oil, gasoline, coal, aluminum, copper, gold, silver, palladium, and platinum.

According to an exemplary embodiment of the present invention, the product may include a pharmaceutical product or pharmaceutical packaging, which may be marked to generate the marked product 201. The pharmaceutical product or pharmaceutical packaging may include a pharmaceutical label, a pharmaceutical packaging insert, and a pharmaceutical packaging cap.

According to an exemplary embodiment of the present invention, the product may include a manufactured good, lumber, furniture, plastic, metal, glass, wood, an adhesive, a painting, a painting and frame, a monetary paper, a coin, a passport, an identification card, a credit card, an ATM card, a metal machined part, a medical device, a sports good, paper, and packaging, which may be marked to generate the marked product 201.

The marked product 201 may be introduced to the stream of commerce, and may be delivered to one or more foreign countries (e.g., the first foreign country 103, the second foreign country 106 and/or the third foreign country 108) for processing to generate a value added product 211.

According to an exemplary embodiment of the present invention, the marked product 201 may be sent directly to a single foreign country (e.g., the first foreign country 103) and then returned to the home country 101 after the processing/value added steps are performed. Alternatively, the marked product 201 may be sent to the first foreign country 103 for processing and then sent to one or more additional foreign countries (e.g., the second foreign country 106 and/or the third foreign country 108) for additional processing and to generate the value-added product 211. For example, the marked product 201 may undergo a first processing/value-added step 104 in the first foreign country 103, a second processing/value-added step 105 in the second foreign country 106, and/or a third processing/value-added step 107 in the third foreign country 108 prior to being returned to the home country 101. That is, the value added product 211 may include the marked product 201 originating in the home country 101.

According to an exemplary embodiment of the present invention, the processing or value-added steps (e.g., 104, 105 and/or 107) may include generating a fully or partially completed product. For example, the marked product 201 may be a circuit board, which is assembled in the home country 101 and then is processed into a computer or another electronics product to form the value-added product 211 (e.g., the computer including the circuit board) in one or more foreign countries (e.g., the first foreign country 103, the second foreign country 106 and/or the third foreign country 108). Thus, a partially assembled product originating in the home country 101 may be processed into a fully completed product which is then sent back to the home country 101.

Prior to entering the home country 101, the value added product 211 may pass through the customs inspection location 110. For example, the value-added product 211 entering the United States may pass through a U.S. Customs and Border Protection location prior to entering the United States. One role of the customs inspection location 110 may be to perform the tariff determination 109. According to an exemplary embodiment of the present invention, one or more taggants or markers (e.g., a nucleic acid marker) included in the marked product 201 may be authenticated 112 at the customs inspection location 110. For example, the authentication may occur prior to or at the time of assessing any tariff(s) that may be due on products entering the country. That is, tariff(s) may be due on foreign derived raw materials or foreign derived products, but not on products or raw materials that originate in the home country. Thus, it may be determined that some portion or some percentage of the value-added product 211 is not subject to one or more tariff(s) because the value-added product 211 includes one or more products which originated in the home country 101.

According to an exemplary embodiment of the present invention, the one or more tariff(s) may not be due on a percentage be weight of the product or might not be due on a percentage of the value of the product. For example, a tariff may only be due on the increase in value from the marked product to the value-added product.

The authenticated value-added product 211 including the marked product 201 may then pass into the home country 101 after passing through the customs inspection location 110.

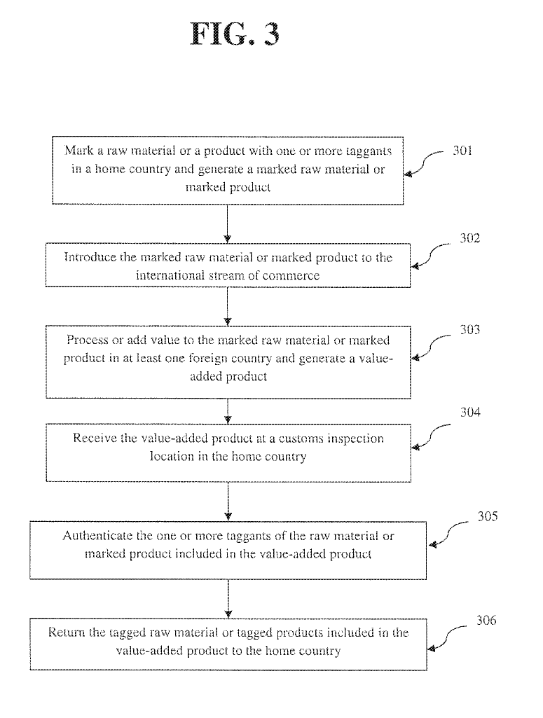

FIG. 3 illustrates an exemplary method of authenticating a taggant of a marked raw material or a marked product included in a value-added product according to an exemplary embodiment of the present invention.

Referring to FIG. 3, a method of authenticating a taggant of a marked raw material or a marked product included in a value-added product returning from the international stream of commerce may include marking the raw material or the product with one or more taggants in a home country and generating a marked raw material or marked product 301. The marked raw material or marked product may be introduced into the international stream of commerce 302. The marked raw material or marked product may be processed and value may be added to the marked raw material or marked product in at least one foreign country to generate a value-added product 303. The value-added product may be received at a customs inspection location in the home country 304. The one or more taggants of the raw material or marked product included in the value-added product may be authenticated 305. The marked raw material or marked product include in the value-added product may be returned to the home country 306. That is, the value-added product, which includes the raw material or product originating in the home country, may enter the home country.

The authentication may occur at the customs inspection location in the home country, and/or at the time of inspecting the value-added product at the customs inspection location. For example, the authentication may occur prior to or at the time of assessing any tariff(s) that may be due on products entering the country. That is, tariff(s) may be due on foreign derived raw materials or foreign derived products, but not on products or raw materials that originate in the home country.

Marking Products or Raw Materials with DNA

### Marker Molecules

In an exemplary embodiment of the present invention, a marker molecule to be deposited, linked, attached or bonded to product, raw material or other article may be a biomolecule (e.g., a nucleic acid marker). The marker molecule may be an inorganic molecule and may include one or more metals, non-metals or rare earth metals. The biomolecule may be a protein, a peptide, a nucleic acid, a vitamin, or a protein-DNA complex. The nucleic acid may comprise, for example, RNA, DNA, an RNA-DNA complex, single stranded DNA or double stranded DNA. The nucleic acid may be any suitable size, for example, the nucleic acid may be in a range of about 50 base pairs to about 1000 base pairs. The nucleic acid may comprise any suitable natural or non-natural DNA sequence such as a synthetic DNA sequence that is not a natural DNA sequence. The non-natural DNA sequence may be formed by digesting and re-ligating naturally or non-naturally occurring DNA. The DNA may be from any source, such as for instance, animal or plant DNA. The DNA may be derived from bacteria, viruses, fungi, or synthetic vectors or fragments or any combination thereof. The nucleic acid may comprise a non-naturally occurring DNA sequence formed by, for example, digesting and relegating animal or plant DNA. The nucleic acid may include synthetic DNA, semi-synthetic DNA of a combination of synthetic and semi-synthetic DNA. The nucleic acid may comprise nuclear, mitochondrial or chloroplast DNA or total genomic DNA.

In an exemplary embodiment of the present invention, the nucleic acid marker may be derived from any suitable DNA source, such as for instance, DNA extracted from a plant source. The nucleic acid marker including DNA may interchangeably be referred to as a DNA taggant. The extracted DNA may be specifically or randomly digested and ligated to generate artificial nucleic acid sequences which are unique to the world. The digestion and ligation of the extracted DNA may be completed by standard restriction digestion and ligation techniques known to those skilled in the art of molecular biology. Digestion may be performed randomly or site-specifically, for example by random or site specific nucleases. The nucleic acid fragments resulting from digestions may be specifically or randomly rearranged to form new nucleic acid sequences (e.g., non-natural nucleic acid sequences). The sequence of the nucleic acid marker can be of any suitable length, for instance the sequence of the nucleic acid marker can be a sequence of from about 5 to about 5000 bases or a sequence from about 20 to about 1000 bases.

In an exemplary embodiment of the invention, the nucleic acid marker may include activated DNA, or any suitable functionalized DNA, for example, an alkaline pH activated DNA (see below). The method may include depositing the nucleic acid marker onto the surface of the article or into a liquid for binding, linking or attaching of the activated nucleic acid marker to the article, for example, onto a surface of the article or a portion of the surface of the article. The nucleic acid marker may be incorporated into the material or a portion of the material from which the article is formed. The alkaline pH activated nucleic acid marker including alkaline activated DNA may be bound to a material, such as, for instance, cotton, wool, nylon, plastic, metal, glass, wood, or printing ink. Alkaline activation of a nucleic acid marker is discussed in more detail below.

Nucleic acid markers may include nucleic acids from animals, plants, bacteria, viruses, fungi, or synthetic vectors or fragments or any combination thereof. The nucleic acid marker may be any suitable nucleic acid, such as for instance, a synthetic non-natural DNA, a semi-synthetic DNA derived from natural and synthetic sequences or a rearranged natural DNA sequence derived by cleavage and ligation of the cleavage fragments in a new non-natural sequence.

The nucleic acid marker may have a specific template sequence and/or a specific template length, so that when polymerase chain reaction (PCR) procedures are performed, PCR primers may be any specific primer pairs with a complementary nucleic acid sequence which can bind nucleic acids of the nucleic acid marker template. There may be a relatively low concentration of nucleic acids in the nucleic acid marker and the nucleic acids may be amplified by techniques well known to those skilled in the art of molecular biology.

The nucleic acid marker may be mixed into solution with water or any desired aqueous solution or buffer to form the solution comprising the nucleic acid marker for use in the methods of the invention. For example, nucleic acids may be mixed with water to form the solution comprising the nucleic acid marker. The solution comprising the nucleic acid marker may be mixed at any desired concentration to mark the article. For example, the concentration of nucleic acid to solvent may be approximately 1 attogram/milliliter (10-18 g/ml), 1 femptogram/milliliter (10-15 g/ml), 1 picogram/milliliter (10-12 g/ml), 1 nanogram/milliliter (10-9 g/ml) or 1 microgram/milliliter (10-6 g/ml). Alternatively, the concentration of nucleic acid in the solution may be in a range from approximately 1 attogram/milliliter (10-18 g/ml) to approximately 1 microgram/milliliter (10-6 g/ml). The solution comprising the nucleic acid marker may include more than one nucleic acid marker.

It will also be appreciated by those of skill in the art that the nucleic acid marker may be combined with one or more optical reporters, for instance, an infrared marker. For example, the optical reporter may be chemically linked to the nucleic acid marker or the optical reporter may be mixed into the solution comprising the nucleic acid marker. The optical reporter may be, for instance, an upconverting phosphor or a fluorophore. The nucleic acid marker and the optical reporter can be mixed in a dyeing process. The combination or mixture of the nucleic acid marker and the optical reporter may be applied to one or more articles, such as for instance, fibers or fibrous materials. The fibers or fibrous materials may be materials suitable for being combined to form textiles. The marked fibers may then be blended with one or more unmarked fibers to generate a marked textile. The blending of the marked fibers with the unmarked fibers may be performed during ginning, before opening, during opening, before blending, or during blending. The fibers may be raw fibers, and may be marked during or after scouring. Raw fibers (e.g., raw cotton fibers or raw wool fibers) may refer to fibers that have been ginned, or ginned and scoured. For example, the raw fibers that have been separated from cotton plant material by ginning, but that have not yet been scoured may include small plant parts and foreign matter that is not removed by the ginning process.

### Activation of Nucleic Acids

Nucleic acids (e.g., DNA) can be activated to enhance binding between the nucleic acid and an article to be marked by methods well known in the art. Activating the nucleic acid may make the nucleic acid physically or chemically reactive with the surface of the article to be marked (e.g., by rendering the nucleic acid capable of ionically or covalently bonding to an available group on the surface of the article). For example, the nucleic acid may be activated by exposure to alkaline conditions. Alkaline activation of nucleic acids is discussed in more detail below.

A reactive functional group may be bound to the nucleic acid to facilitate binding between the nucleic acid and the article to be marked. The reactive functional group may be bound to the nucleic acid through a process of alkaline activation of the DNA molecule (described in more detail below). The reactive functional group may be capable of covalently binding to an available group on at least a portion of the article to be marked. The reactive functional group may immobilize the nucleic acid to the article.

The nucleic acid may be bound to at least a portion of the surface of the article by a chemical linker bound to a reactive functional group. For example, the chemical linker may include a chain of carbon atoms with a reactive functional group at an end of the chain of carbon atoms. The end of the chain of carbon atoms opposite to the reacting functional group may be covalently bound to the nucleic acid. The reactive functional group may be activated to covalently bind with an available group on the surface of the article. Activation of the reactive functional group may be performed by exposure to alkaline conditions. Alkaline activation is discussed in more detail below. The solution comprising the nucleic acid marker may include an activated nucleic acid as described herein.

### Alkaline Activation of Nucleic Acids

The hydroxide anion has the chemical formula: OH−. It consists of an oxygen atom and a hydrogen atom held together by a covalent bond, and carries a negative electric charge. It is an important constituent of water. It functions as a base, as a ligand, a nucleophile, and a catalyst. The hydroxide ion forms salts, some of which dissociate in aqueous solution, liberating solvated hydroxide ions.

In organic chemistry, the hydroxide ion can act as a catalyst or as a nucleophilic reagent. An hydroxyl (OH) group, is present in alcohols, phenols, carboxylic acids and related functional groups.

Water is at equilibrium with its component ions:

[H2O]<=>[H+]+[OH−]

Water contains a concentration of 10-7 M [H+] ions. This is expressed as water having a pH of 7.0 on the logarithmic scale.

Strong alkalis are almost completely dissociated. Thus, the strong alkali, sodium hydroxide is essentially completely dissociated in an aqueous solution.

[NaOH]=>[Na+]+[OH−]

Water is only partly dissociated and has a fixed dissociation constant K according to the formula:

$$K = \frac{[ {H}^{+} ] + [ {OH}^{-} ]}{[ {H}_{2} O ]}$$

Thus, an increase in the concentration of the OH− ion forces the lowering of the concentration of H+ ions, by covalent binding to produce water molecules. Using this formula the concentration of [H+] and thus the pH of a sodium hydroxide solution can be readily estimated:

1.0 M NaOH contains 10-14 M [H+] ions, i.e. has a pH of 14.0;

0.1 M NaOH contains 10-13 M [H+] ions, i.e. has a pH of 13.0;

0.01 M NaOH contains 10-12 M [H+] ions, i.e. has a pH of 12.0;

0.001 M NaOH contains 10-11 M [H+] ions, i.e. has a pH of 11.0;

0.0001 M NaOH contains 10-10 M [H+] ions, i.e. has a pH of 10.0;

0.00001 M NaOH contains 10-9 M [H+] ions, i.e. has a pH of 9.0; and so on.

Alkaline extraction of DNA from cells of organisms takes advantage of the alkali-stable nature of DNA. Cell membranes are disrupted by treatment with alkali, releasing the cellular contents, and melting the double-stranded the total genomic DNA, including nuclear and mitochondrial DNA as the single stranded DNA forms. These DNA strands readily re-hybridize, snapping back to their original double stranded helical structure that can be isolated from the alkali-treated cellular millieu.

Alkali treatment of DNA may activate the DNA for covalent binding. Alkaline conditions may lead to ionization of the free hydroxyls at the 3′ ends of the DNA strands. The negatively charged —O— group produced at the 3′ end of the DNA is a strong nucleophile, reactive with positively charged groups to form stable covalent bonds, stably binding the DNA.

The invention provides methods of binding of a nucleic acid (e.g., DNA) to an article. The method may include exposing the nucleic acid to alkaline conditions, and contacting the nucleic acid to the article. The nucleic acid bound to the article may be available for binding by hybridization probes, PCR amplification and DNA sequencing methods.

In one embodiment, the alkaline conditions are produced by mixing the DNA with an alkaline solution having a high pH, for instance the pH of the alkaline solution can be a pH of about 9.0 or higher; a pH of about 10.0 or higher; a pH of about 11.0 or higher; a pH of about 12.0 or higher; a pH of about 13.0 or higher; a pH of about 14.0 and contacting the DNA that has been exposed to the alkaline conditions with the substrate. In one embodiment, the alkaline solution is a solution of a hydroxide of an alkali metal.

An exemplary embodiment of the present invention provides a method of binding a nucleic acid marker (e.g., a nucleic acid marker including deoxyribonucleic acid) to the article, the method including exposing the DNA to alkaline conditions, wherein the alkaline conditions are produced by mixing the DNA with an alkaline solution, and contacting the DNA that has been exposed to the alkaline conditions with the article; wherein the alkaline solution is a solution of a hydroxide of an alkali metal and the alkali metal is selected from the group consisting of lithium (Li), sodium (Na), rubidium (Rb), and cesium (Cs).

An exemplary embodiment of the invention provides a method of binding the nucleic acid marker (e.g., a nucleic acid marker including DNA) to the article, the method including exposing the DNA to alkaline conditions, wherein the alkaline conditions are produced by mixing the DNA with an alkaline solution, and contacting the DNA that has been exposed to the alkaline conditions with the article; wherein the alkaline solution is a solution of an alkali metal hydroxide, wherein the alkali metal hydroxide is selected from the group consisting of lithium hydroxide (LiOH), sodium hydroxide (NaOH) and cesium hydroxide (CsOH). In one embodiment, the alkali metal hydroxide is sodium hydroxide (NaOH).

An exemplary embodiment the invention provides a method of binding the nucleic acid marker (e.g., a nucleic acid marker including DNA) to the article, the method including exposing the DNA to alkaline conditions, and contacting the DNA that has been exposed to the alkaline conditions with the article; wherein the alkaline conditions are produced by mixing the DNA with a solution of an alkali metal hydroxide, wherein the alkali metal hydroxide solution having a concentration of from about 1 mM to about 1.0 M. In another embodiment the alkaline conditions are produced by mixing the DNA with a solution of an alkali metal hydroxide, the alkali metal hydroxide solution having a concentration of from about 10 mM to about 0.9 M. In still another embodiment the alkaline conditions are produced by mixing the DNA with a solution of an alkali metal hydroxide, the alkali metal hydroxide solution having a concentration of from about 0.1 M to about 0.8 M. In yet another embodiment the alkaline conditions are produced by mixing the DNA with a solution of an alkali metal hydroxide, the alkali metal hydroxide solution having a concentration of from about 0.4 M to about 0.8 M. In still another exemplary embodiment the alkaline conditions are produced by mixing the DNA with a solution of an alkali metal hydroxide, the alkali metal hydroxide solution of about 0.6 M.

An exemplary embodiment of the invention provides a method of binding of the nucleic acid marker (e.g., a nucleic acid marker including DNA) to the article, wherein the method includes exposing the DNA to alkaline conditions and contacting the alkaline exposed DNA to the article, wherein the DNA is mixed with an alkaline solution having a pH from about 9.0 to about 14.0 and incubated at a temperature of from about 0° C. to about 65° C. to produce the alkaline conditions. Alternatively, the incubation temperature may be from about 5° C. to about 55° C., or from about 10° C. to about 45° C., or from about 15° C. to about 35° C., or from about 15 C to about 22° C. to produce the alkaline conditions. In another exemplary embodiment the alkaline conditions are produced by mixing the DNA with an alkali metal hydroxide solution having concentration of from about 0.1 M to about 1.0 M and incubating the mixture for a period of from about 1 minute to about 6 hours at a temperature of from about 10° C. to about 45 C, or from about 15° C. to about 25° C. to produce the alkaline conditions. In another exemplary embodiment the alkaline conditions are produced by mixing the DNA with an alkali metal hydroxide solution having concentration of about 0.6 M and incubating the mixture for a period of from about 1 minute to about 6 hours at a temperature of from about 15° C. to about 35° C., or from about 18° C. to about 22° C. to produce the alkaline conditions.

An exemplary embodiment of the invention provides a method of binding a nucleic acid marker (e.g., a nucleic acid marker including DNA) to an article, the method includes exposing the DNA to alkaline conditions, wherein the alkaline conditions are produced by mixing the DNA with an alkaline solution having a high pH, incubating the mixture and then neutralizing the alkaline solution and contacting the neutralized solution containing the DNA that has been exposed to the alkaline conditions with the article. In an exemplary embodiment, the alkaline solution is a solution of a hydroxide of an alkali metal selected from the group consisting of lithium (Li), sodium (Na), rubidium (Rb), and cesium (Cs).

An exemplary embodiment of the invention provides a method of binding a nucleic acid marker (e.g., a nucleic acid marker including DNA) to an article, the method includes exposing the DNA to alkaline conditions, and contacting the DNA that has been exposed to the alkaline conditions with the article; wherein the alkaline conditions are produced by mixing the DNA with an alkali metal hydroxide solution, and adding a molar excess of a polyionic polymer. The polyionic polymer can be any suitable polyionic polymer. In an exemplary embodiment of the present invention, the polyanionic polymer is a polyamino acid. The polyamino acid can be a homopolymer of a natural amino acid such as L-lysine, or a homopolymer of a non-naturally occurring amino acid, such as for instance D-lysine. In an exemplary embodiment, the polyamino acid homopolymer is selected from the group consisting of polyputrescine, polycadaverine, polyspermidine, and polylysine.

According to an exemplary embodiment of the invention, the nucleic acid marker (e.g., a nucleic acid marker including DNA) can be mixed with a solution of any suitable high pH buffer to produce the alkaline conditions. The high pH buffer can be any suitable high pH buffer with a pKa in a range of from about 9.0 to about 11.0 or higher. In an exemplary embodiment, the pH of the high pH buffer can be, for example, a pH of about 9.0 or higher; a pH of about 10.0 or higher; or a pH of about 11.0 or higher. For example, in an exemplary embodiment, DNA can be mixed with a suitable high pH buffer such as CABS (4-[cyclohexylamino]-1-butanesulphonic acid) with a useful pH range of about 10.0-11.4 (at 25° C.). and a pKa of about 10.70 (at 25° C.), Product No. C5580-Sigma Aldrich, St. Louis, Mo.; CAPS (N-cyclohexyl-3-aminopropanesulfonic acid) with a useful pH range of about 9.7-11.1 (at 25° C.)., a pKa of about 10.56 (at 20° C.), a pKa of about 10.40 (at 25° C.) and a pKa of about 10.02 (at 37° C.), Sigma Aldrich Product Nos. C6070 and C2632; AMP (2-amino-2-methyl-1-propanol) with a useful pH range of about 9.0-10.5 (at 25° C.)., a pKa of about 9.70 (at 25° C.), Sigma Aldrich Product Nos. A9199 and A9879; CAPSO (N-cyclohexyl-2-hydroxyl-3-aminopropanesulfonic acid) with a useful pH range of about 8.9-10.3 (at 25° C.), a pKa of about 9.60 (at 25° C.), a pKa of about 9.43 (at 37° C.), Sigma Aldrich Product Nos. C2278 and C8085; CHES (2-(N-cyclohexylamino) ethanesulphonic acid) with a useful pH range of about 8.60-10.0 (at 25° C.)., a pKa of about 9.55 (at 20° C.), a pKa of about 9.49 (at 25° C.) and a pKa of about 9.36 (at 37° C.), Sigma Aldrich Product Nos. C2885 and C8210; AMPSO (3-[(1,1-dimethyl-2-hydroxyethyl)amino]-2-hydroxypropanesulfonic acid) with a useful pH range of about 8.3-9.7 (at 25° C.)., a pKa of about 9.00 (at 25° C.), a pKa of about 9.10 (at 37° C.), Sigma Aldrich Product Nos. A6659 and A7585, to produce the alkaline conditions.

### Generating a Solution Comprising a Nucleic Acid Marker for Textile Applications

The solution comprising the nucleic acid marker may be formed by mixing the nucleic acid in water. A concentrated solution of nucleic acid marker may be mixed with water to form the solution comprising the nucleic acid marker before the solution comprising the nucleic acid marker is deposited onto the article. The nucleic acid marker may be alkaline activated. For example, the nucleic acid marker may be exposed to the alkaline conditions discussed in detail above. An alkaline activator may be provided and the alkaline activator may be mixed with the solution comprising the nucleic acid marker to form an activated nucleic acid marker. The solution comprising the nucleic acid marker may be an aqueous solution comprising the nucleic acid marker. The solution comprising the nucleic acid marker may comprise any suitable working solution, such as an aqueous solution, which may include a buffer.

In an exemplary embodiment of the present invention, the solution comprising the nucleic acid marker may comprise a non aqueous solvent (e.g., polyurethane or silicone). The solution comprising the nucleic acid marker and the working solution may be mixed to form the solution comprising the nucleic acid marker according to the methods described in U.S. Pat. No. 7,115,301.

According to an exemplary embodiment of the invention, a media may be selected that is used as a topical treatment for a fibrous material. The media may be mixed with the nucleic acid marker to generate the solution comprising the nucleic acid marker suitable for topical treatment of the article. The solution comprising the nucleic acid marker may then be topically applied to the article (e.g., a fibrous material). The marked fibrous material may be generated by causing the nucleic acid marker to adhere to the fibrous material. The media suitable for topical treatment may include colorants, dyes, dyeing auxiliaries, print pastes, softeners, lubricants, antistatic agents, water repellants, antimicrobial agents, wetting agents, leveling agents, or water.

According to an exemplary embodiment of the invention, the media may be a viscous spinning solution for fiber spinning. The viscous spinning solution may be mixed with the nucleic acid marker to generate a viscous dope including the nucleic acid marker. The viscous dope may then be extruded through an opening in a spinneret to form the marked fiber. The marked fiber may then be solidified and can then be used in the textile manufacturing process. According to this exemplary method the solution comprising the nucleic acid marker may be embedded in the fiber.

According to an exemplary embodiment of the invention, the nucleic acid may be mixed with a water insoluble media to generate the solution comprising the nucleic acid marker. Firstly, the nucleic acid may be dissolved in a water soluble solution. The method then proceeds to dissolve the water insoluble media in a solvent. An intermediate solution is then used to mix the water soluble solution having the nucleic acid marker with the water insoluble media. The resulting solution comprising the nucleic acid marker is then applied to the desired article. By way of example and not of limitation, the intermediate solution used to generate the solution comprising the nucleic acid marker may include an organic solvent such as ethanol, acetone, chloroform or other such organic mixtures.

### Application of the Nucleic Acid Marker to an Article

In exemplary embodiments of the invention, the article may include a textile, a fiber, cotton, raw cotton, ginned cotton, a cotton blend, wool, yarn, cashmere, a synthetic fabric and a synthetic fabric blend. The article may be, for example, any natural material, fabric or raw material capable of being treated with the solution comprising the nucleic acid marker. The solution comprising the nucleic acid marker may be applied to fibers, yarns, sewing thread, fabrics, non-woven materials, and any product made from fibrous materials, such as a textile including wool or cotton fibers. The article may be any consumer product capable of being treated with the solution comprising the nucleic acid marker.

According to an exemplary embodiment of the present invention, the alkaline activated nucleic acid marker may be applied to raw cotton fibers by spraying a solution including the activated nucleic acid marker onto the raw cotton fibers to mark the raw cotton fibers.

In an exemplary embodiment, the solution comprising the nucleic acid marker may be dried onto the article or absorbed into a material used to make the article. For example, the article may be a textile article including cotton or wool and the solution comprising the nucleic acid marker may be dried on the textile article. The solution comprising the nucleic acid marker may be dried by any suitable drying process, for example, air drying, oven drying, IR drying, or UV curing. Fibers may be any substance, natural or manufactured, with a high length-to-width ratio and with suitable characteristics for being processed into fabric in which the smallest component is hairlike in nature and can be separated from a fabric. Natural fibers may be those that are in a fiber form as they grow or develop and may be from animal, plant, or mineral sources, for example. Manufactured fibers (e.g., synthetic fibers) may be made from chemical compounds produced in manufacturing facilities. The manufactured fiber may be, for instance, Rayon or nylon.

Yarns may be an assemblage of fibers that are twisted or laid together so as to form a continuous strand that can be made into textile fabric or a textile article. A yarn may be a continuous strand of textile fibers, filaments, or materials in a form suitable for knitting, weaving, or otherwise intertwining to form a textile fabric. Filament yarns may be made from manufactured fibers, except for a relatively small percentage that is filament silk. Manufactured filament yarns may be made by extruding a polymer solution through a spinneret, solidifying it in fiber form, and then bringing the individual filaments together with or without a twist. Spun yarns may be continuous strands of staple fibers held together by a mechanism such as a mechanical twist that uses fiber irregularities and natural cohesiveness to bind the fibers together into one yarn.

Sewing thread may be a yarn intended for stitching materials together using machine or hand processes. Fabric may be a flexible planar material constructed from solutions, fibers, yarns, or fabrics, in any combination. A fabric may be a pliable, flat structure that can be made into two- or three-dimensional products that require some shaping and flexibility. Fabrics can be made from a wide variety of starting materials, such as for instance, solutions, fibers, yarns, “composite” fabrics, fiberglass or carbon fiber. For fabrics made from yarns, the fabric may be a woven or knitted fabric. Woven fabrics may be made with two or more sets of yarns interlaced at right angles. Knitting is a process which may form a fabric by the interlooping of one or more sets of yarns. Fabrics from solutions may include films in which the films are made directly from a polymer solution by melt extrusion or by casting the solution onto a hot drum. Composite fabrics are fabrics that combine several primary and/or secondary structures, at least one of which may be a recognized textile structure, into a single structure. Some fabrics may be made directly from fibers or fiber forming solutions without processing of fibers into a yarn. These nonwoven structures may include textile-sheet structures made from fibrous webs, bonded by mechanical entanglement of the fibers or by the use of added resins, thermal fusion, or formation of chemical complexes.

In exemplary embodiments of the invention, an article marked by a process is provided. The process may include providing the article and placing the article on any suitable surface for holding the article for deposition of the solution comprising the nucleic acid marker. For example, the article may be placed on a substrate, a surface, such as a platform, which may be a moving platform, or a conveyor belt. The method of marking the article may include conveying the article along the conveyor belt in the direction of the delivery mechanism positioned at a location along the conveyor belt. The delivery mechanism may comprise one or more outlets. The method of marking the article may include depositing the solution comprising the nucleic acid marker onto the article through the one or more outlets of the delivery mechanism and thereby marking the article.

An exemplary embodiment of the invention provides a device for marking an article including a conveyor belt adapted to convey an article in a direction of a delivery mechanism positioned at a location along the conveyor belt. The conveyor belt may be of any height, width, length or other desired dimensions to accommodate the article to be marked. The conveyor belt may be adapted to move in any desired direction. The conveyor belt may be motorized or manually operable. The conveyor belt may convey the article at a variety of speeds. The speed of the conveyor belt may be adjusted either manually or automatically. The conveyor belt may be controlled by a computer system. The conveyor belt speed may be adjusted according to, for example, a flow rate, a flow pressure or a deposition rate of the solution comprising the nucleic acid marker.

The delivery mechanism may include one or more outlets. The number of outlets may vary according to, for example, the amount of solution comprising the nucleic acid marker that is deposited on the article. The number of outlets may vary according to the size of the conveyor belt or the speed of the conveyor belt. The size of the one or more outlets may be individually and/or collectively adjustable in order to regulate, for example, the flow rate, flow pressure or a deposition rate of the solution comprising the nucleic acid marker. The position of the one or more outlets on the delivery mechanism may be adjustable so that the one or more outlets can be moved. The direction that the one or more outlets face may adjustable, for example, to adjust the direction that the solution comprising the nucleic acid marker exits the one or more outlets. The shape of the one or more outlets may be any suitable shape to output a solution comprising the nucleic acid marker. For example, the one or more outlets may be formed in a cone or cylinder shape. The one or more outlets may be adapted to provide a mist with the solution comprising the nucleic acid marker onto the article. The one or more outlets may be adapted to provide a continuous, non-continuous or intermittent spray onto the article.

The delivery mechanism may be adapted to deposit the solution comprising the nucleic acid marker through the one or more outlets onto the article and marking the article with the solution comprising the nucleic acid marker. The delivery mechanism may be positioned at any suitable region along the conveyor belt. For example, the delivery mechanism may cover a width of the conveyor belt. The delivery mechanism may be positioned at any desired angle to deposit the solution comprising the nucleic acid marker onto the article. For example, the delivery mechanism may be suspended above the conveyor belt or along the side of the conveyor belt. More than one delivery mechanism may be positioned at more than one location along the conveyor belt. The delivery mechanism may include one or more reservoirs, and the reservoirs may store the solution comprising the nucleic acid marker.

In an exemplary embodiment of the invention, the one or more outlets may be disposed on a spray bar positioned to deliver the solution comprising the nucleic acid marker onto the article. The spray bar may be adapted to deposit the solution comprising the nucleic acid marker through the one or more outlets onto the article and marking the article with the solution comprising the nucleic acid marker. The spray bar may be positioned at any region of the platform of the conveyor belt. The spray bar may be positioned at any desired angle to deposit the solution comprising the nucleic acid marker on the article. More than one spray bar may be positioned at more than one location along the conveyor belt. The spray bar may be operatively linked to one or more reservoirs, and the reservoirs may store the solution comprising the nucleic acid marker.

In an exemplary embodiment of the invention, the device for marking an article may include a regulator associated with the delivery mechanism. The regulator may be adapted to regulate an amount of the solution comprising the nucleic acid marker deposited by the delivery mechanism through the one or more outlets. The regulator may also be at any desired position associated with the delivery mechanism to regulate the amount of solution comprising the nucleic acid marker deposited. For example, the regulator may be positioned along a stream of the solution comprising the nucleic acid marker exiting the delivery mechanism. The regulator may regulate, for example, a flow rate, a flow pressure or a deposition rate of the solution comprising the nucleic acid marker. The regulator may be adjusted manually or automatically. The regulator may be automated, for example, by being monitored and/or adjusted by a computer system.

The regulator may regulate, for example, a flow rate, a flow pressure or a deposition rate of the solution comprising the nucleic acid marker at each individual outlet or may regulate all of the one or more outlets simultaneously. The regulator may regulate the deposition rate of the solution comprising the nucleic acid marker according to the rate of the conveyor belt. For example, if the conveyor belt is moving at a slower relative speed, then the regulator may adjust the deposition rate of the solution comprising the nucleic acid marker to be slower. For example, if the conveyor belt is moving at a relatively high speed, then the regulator may adjust the deposition rate of the solution comprising the nucleic acid marker to keep up with the rate of the conveyor belt. The regulator may be used to adjust the deposition rate of the nucleic acid marker solution appropriate for the number or amount of the articles placed on the conveyor belt.

The regulator may regulate a deposition rate of the solution comprising the nucleic acid marker to achieve a desired water content concentration of the article by regulating an amount of the solution comprising the nucleic acid marker (e.g., an aqueous solution) deposited onto the article. For example, the water content concentration of processed cotton that has not been marked with the solution comprising the nucleic acid marker may generally be maintained at approximately 8.5% w/w of water per total weight of cotton. The water content of processed wool that has not been marked with the solution comprising the nucleic acid marker may generally be maintained at approximately 12% w/w of water per total weight of wool.

In an exemplary embodiment of the invention, the device for marking an article may include a measurement apparatus associated with the delivery mechanism. The measurement apparatus may be adapted to measure an amount of the solution comprising the nucleic acid marker deposited by the delivery mechanism. The measurement apparatus may be located at any desired position associated with the delivery mechanism to measure the amount of solution comprising the nucleic acid marker deposited by the delivery mechanism through the one or more outlets. For example, the measurement apparatus may be positioned along a stream of the solution comprising the nucleic acid marker exiting the delivery mechanism. The measurement apparatus may measure, for example, a flow rate, a flow pressure or a deposition rate of the solution comprising the nucleic acid marker. The measurement apparatus may measure, for example, the flow rate of the solution comprising the nucleic acid marker through an individual outlet. The measurement apparatus may be manually or automatically controlled. The measurement apparatus may be controlled by a computer system. The measurement apparatus may provide a signal to the regulator to allow the regulator to adjust the deposition rate of the solution comprising the nucleic acid marker. The measurement apparatus may provide a signal to the regulator to adjust the deposition rate of the solution comprising the nucleic acid marker onto the article to maintain the desired water content concentration. A computer system may be used to monitor and control the regulator and the measurement apparatus.

### Authentication of a Marked Article

The nucleic acid marker may be used to identify specific characteristics of an article. For example, the nucleic acid marker may be used to determine whether or not a particular article of interest is authentic by determining whether the article of interest is marked with the nucleic acid marker. By way of example and not of limitation, the nucleic acid marker may be used to encode product information, such as, country of origin for the textile material, origin of the final product, information about the manufacturer, plant identification, product identification and any other desired or related data. The encoded information is stored in a database.

The presence of the nucleic acid marker in an article of interest may be detected by using portable scanners and/or lab verification methods that may include for instance PCR or isothermal amplification followed by any suitable specific marker sequence detection method, such as for instance specific amplicon size detection, or specific marker sequence detection by hybridization with a sequence specific probe. The marker once detected, may be compared to the information stored in the database so that the information associated with the marker and goods to which it is applied can be retrieved.

For example, when value added processed goods are returned to the country of origin, the material making up the goods can be interrogated to determine if a taggent is present. If the taggent is found, it may be compared to the information stored in the database to determine the country of origin or other information. The proper tariff can then be applied based on the origin of the material used to manufacture the goods.

The value added processed goods can be interrogated in the field using an in-field detection kit. A sample of the goods can be subjected to the interrogation at the point where a tariff determinations is to be made, such as at a customs site. The in-field detection kit may provide for sample in answer out analysis.

In an exemplary embodiment, the present invention provides a detection system for DNA detection and authentication of a type set forth in commonly assigned U.S. Patent Publication No. 2015/0141264 published May 21, 2015, the contents of which are incorporated by reference herein. The detection system may include: a portable detector configured to detect the presence of a DNA marker encoding unique information of an item of interest from a sample of the item; and an analysis device, which can be any suitable analysis device, such as a stand-alone device, for instance a desktop computer, a laptop computer, a smartphone or a tablet, operatively connected to the portable detector and configured to verify the authenticity of the item by determining whether the DNA marker is present in the sample analyzed by the portable detector.

In one embodiment, the analysis device is configured to verify the authenticity of the item by determining whether the DNA marker is present in the sample analyzed by the portable detector by comparing the sample analyzed to a known sequence and/or amplicon length for the DNA marker stored in a database accessible by the analysis device. In exemplary embodiments, the verifying of the authenticity step of the method of the invention may include comparing the DNA marker sequence analyzed in the sample to a sequence of the DNA marker stored in a database accessible to or via the analysis device. In other exemplary embodiments, the verifying of the authenticity step of the method of the invention may include comparing the DNA amplicon lengths generated by the DNA amplification of the analysis device to amplicon length information stored in a database accessible to or via the analysis device.

In an exemplary embodiment, the analysis device is configured to verify the authenticity of the item by determining whether the DNA marker is present in the sample analyzed by the portable detector by recognizing a micro-array pattern to determine that the DNA marker is present. Alternatively, the analysis device is configured to verify the authenticity of the item by determining whether the DNA marker is present in the sample analyzed by a portable detector by detecting the presence of amplified or hybridized DNA detected by a complementary nucleic acid sequence.

In one embodiment, verifying of the authenticity of the article of interest is performed using an analysis device operatively connected to a portable DNA sequencing device. The analysis device can be any suitable analysis device, such as for instance a desktop computer, a laptop computer, a smartphone, a tablet, or the like. In exemplary embodiments, the analysis device is encryption and password protected against unauthorized use.

In one embodiment, the verifying of authenticity includes comparing the DNA marker sequence analyzed in the sample to a known sequence of the DNA marker stored in a database accessible via the analysis device. The DNA sequence can be stored locally in a stand-alone server or a network server operatively connected to the analysis device by a wired or a wireless connection. In an exemplary embodiment, the analysis device can be modularized. The results of the verification of authenticity can be processed and stored on a local server or a network server. The sequence of the DNA marker can be any suitable unique sequence. In one embodiment, the sequence of the DNA marker is a sequence of from about 20 to about 1000 bases.

The known sequence of said DNA marker can be any unique sequence for uniquely identifying the item, such as, for instance, a sequence of from about 20 to about 1000 bases. The item marked with the DNA marker can also be marked with a detectable reporter. In one embodiment, the detectable reporter and the DNA marker are in the same location on the item, so that the detectable reporter can be used as a sample identifier for the position of the DNA marker. Alternatively, the detectable reporter and the DNA marker can be associated with each other, so that obtaining a sample of the detectable reporter can be used as a guide to obtain a sample of the DNA marker for authentication and validation. In an exemplary embodiment, the detectable reporter of the method is selected from a chemical reporter, a digital reporter or a peptide reporter. In an exemplary embodiment, the detectable reporter is a digital reporter is a barcode.

The sequence detection system can include an analysis device that is encrypted and/or password protected against unauthorized use. The analysis device can be linked to a server by a wireless connection or a hard wired connection. The server can be a stand-alone server or a network server. In one embodiment, the analysis device may be modularized. In one embodiment, the data verifying the authenticity is processed and stored on the stand-alone server or the network server.

In one embodiment, the marked item to be authenticated by the system of the invention is selected from a microchip, a label, a badge, a logo, a printed material, a textile or a commodity. In an exemplary embodiment, the item to be authenticated by the system is selected from the group consisting of cash, a currency note, a coin, a gem, an item of jewelry, a musical instrument, a passport, an antique, an item of furniture, artwork, a property deed, a stock certificate, or a bond certificate. The detection system of the invention can be configured to provide authentication data directly to the user via a display, or to a secure location, or headquarters to provide data for communication to a customer, or for storage in a database or printing a report. The authentication data can be read as a display or signal, or alarm.

The analysis device of the method for in-field detection and authentication of DNA can be any suitable DNA analysis device, such as for instance a desktop computer, a laptop computer, a smartphone, or a tablet, etc. These devices may be operatively configured to communicate as a system in a network. The network can be any suitable network, such as for instance a network with one or more connections without limitation, by RF, Wi-Fi, Bluetooth or a hard-wired connection.

### In-Field Detection Instruments

The in-field detection instrument may include a microsystem with sample in-answer out analysis capability. The microsystem may be a self-contained unit that performs all necessary analysis processes without the need for additional lab equipment. The microsystem may by automated, and may only require the addition of the sample to the microsystem and activation of the microsystem to perform the analysis. The in-field detection instrument may be portable or fixed in a single location. Sample in-answer out analysis refers to the ability of the microsystem to perform all analysis steps after transferring the sample to the microsystem and automatically providing a result. The microsystem may be configured to provide detection with a minimum of necessity for monitoring or adjustment by the operator. In an exemplary embodiment, the sample is loaded directly or from a sample collection device configured to mate with a sample port of the microsystem. The microsystem is then activated and the in-field detection instrument provides detection data with operator interaction. Detection data may be stored and/or exported. In the case of detecting the presence of a distinctive marker, a sample suspected of including the distinctive marker may be provided and transferred to the microsystem of the in-field detection instrument, and the microsystem may automatically determine whether or not the distinctive marker is present in the sample. Exemplary microsystems for in-field detection are described in more detail below.

The in-field detection instrument may communicate with a server comprising authenticity data for the article of interest. The server may comprise an authenticity data database storing profile information for a number of distinctive markers associated with a number of articles of interest. Authenticity data may be a unique profile corresponding to the distinctive marker. For example, the distinctive marker may include one or more unique nucleic acid sequences, and the authenticity data may be a digital copy of the unique nucleic acid sequences. An example of a suitable remote authentication server including an authentication database is described in Zorab (WO 01/99063).

### Microfluidic Sample Preparation Devices

A system utilizing microfluidics which are suitable for use in exemplary embodiments of the methods of the present invention is disclosed in Jovanovich et al. (U.S. Pat. No. 7,745,207 B2). Further, nanofluidics as disclosed by Janovich et al. (US2012/0115189 A1) and sample handling as disclosed in Green (U.S. Pat. No. 8,110,397 B2) are useful in additional embodiments of the present invention. A PCR device as described in Green (U.S. Pat. No. 7,170,594 B2) can also be used in exemplary embodiments of the present invention. Analysis methods have been developed using microfluidic systems and fractionation or partitioning of DNA solutions down to about one DNA molecule per droplet to identify polymorphisms in a quantum dot (Lo et al., US2009/0263580 A1). Methods and devices for digital PCR including the use of “droplet-in-oil” technology useful in the practice of embodiments of the present invention are disclosed by Davies et al. (US2010/0092973 A1). Oligonucleotide sequences have been used for the detection of the ricin gene and the ricin toxin (Czajka, US 2006/0240447) and a kit was created to detect this specific DNA in a sample.

According to exemplary embodiments of the present invention, microfluidic devices may be used for extraction, purification and stretching of a DNA sample. For instance, a microfluidic device for extraction, purification and stretching of human DNA from single cells is described in Benitez et al. “Microfluidic extraction, stretching and analysis of human chromosomal DNA from single cells” Lab on a Chip 12.22 (2012): 4848-4854. A microfluidic DNA chip is described in Zhao et al. “Electrochemical DNA detection using Hoechst dyes in microfluidic chips” Current Applied Physics 12.6 (2012): 1493-1496. A direct ultra-fast PCR for forensic genotyping is described in Aboud, “The development of direct ultra-fast PCR for forensic genotyping using short channel microfluidic systems with enhanced sieving matrices” (Jan. 1, 2012). ProQuest ETD Collection for FIU. Paper AAI3541755.

For instance, microfluidic devices generally, and microfluidic capillary electrophoresis devices in particular, are described in Wu, Jinbo, et al. “Extraction, amplification and detection of DNA in microfluidic chip-based assays” Microchimica Acta (2013): 1-21; Liu et al. “Integrated DNA purification, PCR, sample cleanup, and capillary electrophoresis microchip for forensic human identification” Lab on a Chip 11.6 (2011): 1041-1048; U.S. Pat. No. 7,745,207 B2 to Jovanovich et al.; and published US Patent Application US2012/0115189 A1 to Jovanovich et al.

According to exemplary embodiments of the present invention, one or more samples may be provided including one or more target oligonucleotide sequences, and one or more non-target oligonucleotide sequences (e.g., camouflage or decoy sequences). The target oligonucleotides may be purified by separating the target oligonucleotides from the camouflage oligonucleotides. The sample may be washed over a bed of substrates immobilized in a series of microchannels. The substrates may include one or more oligonucleotide probes that are complementary to the target oligonucleotides and are therefore configured to capture the target oligonucleotides through hybridization. The substrate may be any suitable substrate. For example, the substrate may be a magnetic bead with oligonucleotides probes bound thereto. The target oligonucleotides may bind the oligonucleotide probes to form a oligonucleotide-probe conjugate. The substrates (e.g., magnetic beads) bound to the target oligonucleotides may be transferred to a detection module to determine whether each target oligonucleotide is present. The substrates (e.g., magnetic beads) bound to the target oligonucleotides may be transferred to an amplification device or an amplification chamber. Amplified products may be transferred, for example, to a capillary electrophoresis column. Oligonucleotide separation may be performed by the application of an electric field according to standard capillary electrophoresis techniques known in the art. A laser with an optical detector system may be employed to detect the presence of the target oligonucleotides. Capillary electrophoresis and DNA detection techniques are described in Schwartz, Guttman. Separation of DNA by capillary electrophoresis. Beckman, 1995.

### Integrated Microsystems

According to exemplary embodiments of the present invention, a nucleic acid marker may be detected by an integrated microsystem. Integrated microsystems may include, for example, a microfluidic chip, a fully integrated microdevice or a lab on a chip configuration. Integrated microsystems can prepare a sample, amplify a target nucleic acid sequence (if necessary) and detect the presence of one or more target nucleic acid sequences on a single device. All steps can be performed on a self-contained, automated microsystem without the need for performing any manual testing in a lab. For instance, integrated microsystems are described in Wu et al. “Extraction, amplification and detection of DNA in microfluidic chip-based assays” Microchimica Acta (2013): 1-21; Liu et al. “Integrated DNA purification, FOR, sample cleanup, and capillary electrophoresis microchip for forensic human identification” Lab on a Chip 11.6 (2011): 1041-1048; and Ullrich, Thomas, et al. “Competitive Reporter Monitored Amplification (CMA)-Quantification of Molecular Targets by Real Time Monitoring of Competitive Reporter Hybridization” PloS one 7.4 (2012); e35438.

In other exemplary embodiments, the sample containing the nucleic acid taggant can be separated from other sample components, such as by capillary action in a microfluidic channel system of a power-free microchip, and detected by binding to a labelled immobilized hybridization probe which is complementary to at least a portion of the nucleic acid taggant sequence. The nucleic acid taggant can be detected directly or amplified (e.g. by isothermal amplification or by PCR) and detected by laminar flow assisted dendritic amplification (LFDA) using a detectable antibody specific for the label bound to the immobilized hybridization probe and detected with a second antibody in a dendritic cascade reaction, as is well known in the immunoassay art. See for instance: Hosokawa et al. DNA Detection on a Power-free Microchip with Laminar Flow-assisted Dendritic Amplification, Anal. Sci. (2010) 26: 1053-1057.

According to an exemplary embodiment of the present invention, an integrated microsystem may employ an encapsulated microarray system without automated fluorescent labeling and detection. The integrated microarray system can amplify one or more target sequences from a provided sample by using PCR or by isothermal amplification, fluorescently label one or more of the amplified products and detect the presence of the fluorescently labeled products. For instance, a microarray-in-a-tube detection system is described in more detail in Liu et al. “Microarray-in-a-tube for detection of multiple viruses” Clinical chemistry 53.2 (2007): 188-194.

According to an exemplary embodiment, a device for in-field detection of a distinctive marker includes an in-field detection instrument including a microsystem configured to perform sample in-answer out analysis. The in-field detection instrument is configured to analyze a sample to determine the presence of a distinctive marker in the sample. According to an exemplary embodiment, the in-field detection instrument may be configured to perform any suitable chemical or physical characterization method capable of determining one or more distinctive characteristics of the distinctive marker.

According to an exemplary embodiment, the sample collection unit may be configured to be directly coupled to the in-field detection device.

According to an exemplary embodiment, the integrated microsystem may employ a fluorescence background displacement technique. An array of immobilized oligonucleotide capture probes may be disposed in the reaction chamber having the reaction mixture. A solution comprising fluorescently labeled reporter oligonucleotide probes that are complementary to the immobilized oligonucleotide capture probes may also be provided in the reaction chamber. Amplicons generated in the reaction chamber that are complementary to the reporter oligonucleotide probes may compete with the capture probes for binding with the reporter oligonucleotide probes. That is, the reporter probes may each also be complementary to a specific target oligonucleotide sequence. Thus, as the number of copies of a particular amplicon (i.e., target oligonucleotide sequences) increases, the fluorescent signal detected in the corresponding immobilized oligonucleotide capture probe decreases because fewer reporter oligonucleotide probes bind the corresponding capture probe. This may allow simultaneous qualitative and quantitative detection of target nucleic acid sequences included in the oligonucleotide sequences of the sample. Optical images of the capture probe may be obtained at any desired time point, including at baseline before a sample has been added to the reaction mixture. Binding of non-target nucleic acid sequences (e.g., camouflage sequences) in the sample to the reporter probes may be avoided because of the hybridization specificity between the reporter probes and the target nucleic acid sequences. The capture probes may also be configured for direct binding between the target nucleic acid sequence and the capture probe. The reaction mixture holding unbound reporter probes may be displaced so that only bound reporter probes (bound to the capture probes) are detected. The reaction mixture may be displaced through any suitable displacement mechanism. For instance, mechanical fluorescence background displacement techniques are described in Ullrich et al. “Competitive Reporter Monitored Amplification (CMA)-Quantification of Molecular Targets by Real Time Monitoring of Competitive Reporter Hybridization” PloS one 7.4 (2012): e35438.

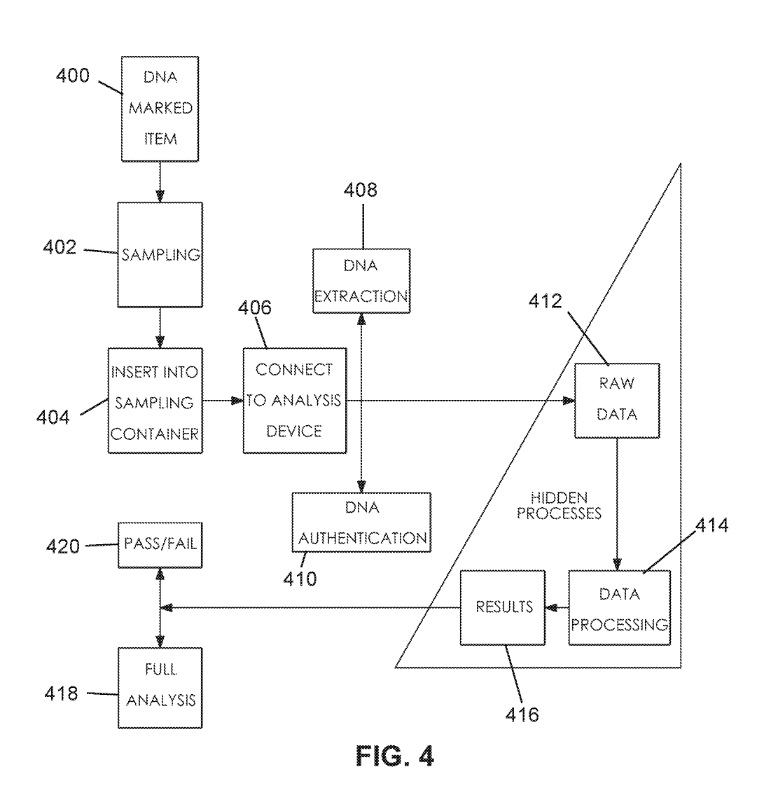

FIG. 4 shows an embodiment of a process according to the present invention. An item is carrying a DNA marker 400 is sampled 402, the sample is transferred to a sampling container 404 which is operatively connected to an analysis device 406. The sample 400 can be subjected to a DNA extraction process step 408, as appropriate for the particular sample and detection and authentication steps 410 are carried out. Raw data 412 is communicated to a data processing unit 414. Results data 416 from the processing unit can then be relayed to a local or remote processor or database for further analysis 418, display, storage or incorporation into a report, such as for instance, a “Pass/Fail” readout 420. Processes inside the triangular region and the rectangular analysis and readout regions are hidden, secure processes.

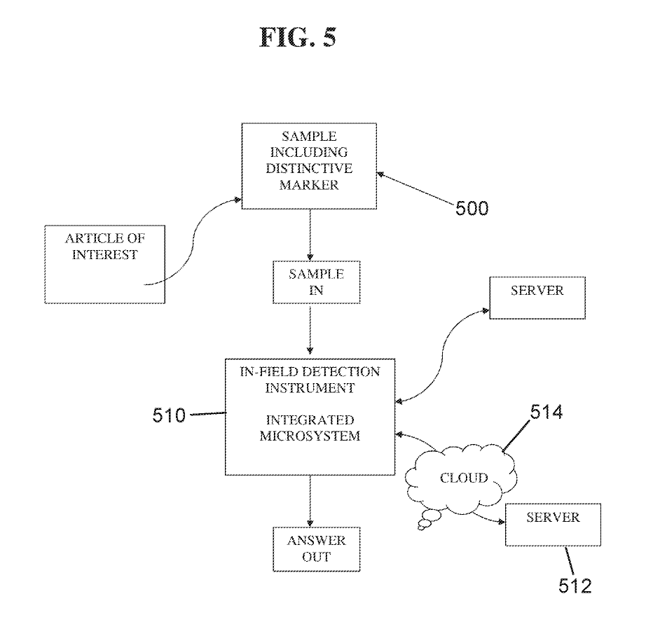

FIG. 5 shows an in-field detection instrument according to an exemplary embodiment of the present invention. A sample 500 is collected from an article of interest. The sample 500 is transferred to the in-field detection instrument. The in-field detection instrument 510 includes an integrated microsystem with sample in-answer out analysis capability to detect the presence of the distinctive marker. The in-field detection instrument 510 may provide a direct answer-out, for example as a readout on a screen or printout. Alternatively, or in addition, the in-field detection instrument may communicate with a server 512 to compare data generated by the in-field detection instrument with data stored in the server. The server may be a free standing server or the server may be in the cloud 514, and the server in the cloud may in turn be linked to a free standing server.

### Example 1

An Integrated Microsystem and Method for Rapid in-Field Detection of a Distinctive Marker

According to an exemplary embodiment of the present invention, the location of a distinctive marker including one or more nucleic acid sequences is identified on an article of interest. A sample of the distinctive marker including the nucleic acid sequences is obtained, and the sample is transferred to an array tube including a custom array chip. The sample is selectively fluorescently labeled and the presence of one or more nucleic acid sequences is determined by microarray analysis.

An array tube (AT) with a custom probe microarray in a microreaction vial may be employed. The array tube may be, for example, an Alere Technologies GmbH ArrayTube with a custom probe microarray (i.e., biochip) integrated into a microreaction vial, which is commercially available (Alere Technologies, Jena, Germany). The custom array includes oligonucleotides that are complementary to nucleic acid sequences making up the distinctive marker that is unique to the article of interest. The custom array chip includes a custom selection of complementary oligonucleotides probes that are spotted onto the array chip. The oligonucleotides probes hybridize with complementary target nucleic acid sequences to detect the presence of the target nucleic acid sequence in a sample.

The probe microarrays are customized for individual applications with oligonucleotide, polynucleotide or protein/peptide probe molecules. Each AT chip with dimensions of 3 mm.times.3 mm can be provided with up to 14.times.14 features including reaction control and reference marker spots.

Depending on the individual assay, nucleic acid as well as protein and peptide based arrays can be manufactured. Each AT is labeled with a unique data matrix code (a two-dimensional bar code) identifying array and related assay. AT testing is performed in combination with the method of precipitation staining, which is suited to both serological and nucleic acid based analysis. Catalytically induced precipitation directly correlates to the amount of target molecules binding to the array. Analysis of the resulting precipitation pattern is done by simple transmission measurements, leading to the effective reduction of the input in optical equipment. Transmission measurement may be taken with an ATR 03 instrument, which is commercially available (Alere Technologies, Jena, Germany) or an ARRAYMATE instrument, which is also commercially available (Alere Technologies, Jena, Germany).

Data is automatically recorded and analyzed directly from the array tube comprising the custom array chip and results are electronically communicated to a desired computer server or stored to a local disk. Data captured from the automated microarray may be automatically processed and compared with a database of known nucleic acid sequence combinations for authentication.

### Example 2

An Integrated Microsystem and Method for Rapid in-Field Detection of a Distinctive Marker

A sample of a DNA marker of interest is provided and transferred to the integrated microsystem device for analysis. The sample includes one or more oligonucleotide sequences. The oligonucleotide sequences may include one or more target oligonucleotide sequences of the distinctive marker and one or more non-target oligonucleotide sequences as camouflage or carrier decoy sequences.

The sample includes a number of oligonucleotide sequences. Target oligonucleotides are purified from the sample (e.g., to remove extraneous or camouflage oligonucleotides sequences) by washing the sample over a bed of magnetic beads immobilized in a series of microchannels. Each magnetic bead has one or more oligonucleotide probes attached thereto which are complementary to one of the target oligonucleotides that is distinctive to the article of interest. Target oligonucleotides are bound to the oligonucleotide probes of the magnetic beads to form a DNA-probe conjugate. This sequence-specific DNA purification improves downstream amplification efficiency by removing background oligonucleotide sequences (i.e., the target oligonucleotides are purified). The sample is moved across the capture channels using a micropump.

The magnetic beads serve as a medium to carry the target oligonucleotides to an isothermal amplification unit for amplification. The magnetic beads including DNA-probe conjugates are pumped into the isothermal amplification unit and the target oligonucleotides are amplified by isothermal amplification.

The amplified products are pumped through a capture gel suitable for post-amplification injection into a capillary electrophoresis column. The amplified products build up in the capture gel to form a sample plug which can be pumped into a capillary electrophoresis column for separation and detection. The sample plug is released (e.g., thermally released) and injected into a capillary electrophoresis column for target oligonucleotide separation and detection of each of the target oligonucleotide sequences that is present. Oligonucleotide separation is performed by the application of an electric field according to standard capillary electrophoresis techniques known in the art. A laser with an optical detector system is employed to detect the presence of the target oligonucleotides included in the sample plug. Capillary electrophoresis and DNA detection techniques are described in Schwartz and Guttman. Separation of DNA by capillary electrophoresis. Beckman, 1995.

A target sequence profile is generated according to the detected target oligonucleotides from the sample. The target sequence profile includes only the detected target oligonucleotide sequences, but excludes the non-target (camouflage) oligonucleotide sequences. Profile data is either transmitted to a remote server or stored on a local disk for comparison with a library of known sequence profiles to determine if the sample has been provided from an authentic article or not.

### Example 3

An Integrated Microsystem and Method for Rapid in-Field Detection of a Distinctive Marker

A sample of a DNA marker of interest is provided and transferred to the integrated microsystem device for analysis. The sample includes one or more oligonucleotide sequences. The oligonucleotide sequences may include one or more target oligonucleotide sequences of a distinctive marker and one or more non-target oligonucleotide sequences as camouflage or carrier decoy sequences.

The integrated microsystem device includes a reaction chamber, a relief chamber, a reaction chamber plunger, a temperature control module, an optical detection module, and a data analysis module. The integrated microsystem device utilizes a mechanical fluorescence background displacement technique, which employs the following principle. An array of immobilized oligonucleotide capture probes is disposed in the reaction chamber. A solution comprising fluorescently labeled reporter oligonucleotide probes that are complementary to the immobilized oligonucleotide capture probes is also provided in the reaction chamber. Amplicons generated in the reaction chamber that are complementary to the reporter oligonucleotide probes compete with the capture probes for binding with the reporter oligonucleotide probes. That is, the reporter probes are each also complementary to a specific target oligonucleotide sequence. Thus, as the number of a particular amplicons (i.e., target oligonucleotide sequences) increases, the fluorescent signal detected in the corresponding immobilized oligonucleotide capture probe decreases because fewer reporter oligonucleotide probes bind the corresponding capture probe. This allows simultaneous qualitative and quantitative detection of target nucleic acid sequences included in the oligonucleotide sequences of the sample.

According to the present example, prior to the introduction of the sample to the reaction chamber, the reporter probes can freely bind to the capture probes and a baseline fluorescent signal can be acquired. Fluorescent signals are acquired by actuating the reaction chamber plunger to press the capture probe array against a side wall of the reaction chamber, at which point the optical detection module can acquire a fluorescent signal. When the reaction chamber plunger is not actuated, the capture probe array is not pressed against the wall of the reaction chamber, and thus the fluorescent signal is not detected by the optical detection module. Actuating the reaction chamber plunger also displaces the reaction mixture holding unbound reporter probes so that only bound reporter probes (bound to the capture probes) are detected.

The provided sample is introduced into the reaction chamber and the target oligonucleotide sequences are amplified by PCR. To detect a fluorescent signal pattern over time, the system is configured to sequentially capture fluorescent signal images after each PCR amplification cycle. The temperature control module is configured to automatically heat and cool the reaction chamber for PCR amplification and the reaction chamber plunger is actuated for fluorescent signal capture after each PCR amplification cycle is completed. However, the reaction mixture may emit a background fluorescent signal which could disrupt capture of the fluorescent signal from the capture probe array. Therefore, when the reaction chamber plunger is actuated, it compresses the reaction chamber and thereby displaces the reaction mixture into the relief chamber, thus removing the reaction mixture from the reaction chamber, thus substantially eliminating the background fluorescent signal. After fluorescent signal capture from the capture probe array, the reaction chamber plunger retracts, the reaction chamber relaxes, and the reaction mixture flows from the relief chamber back into the reaction chamber. This process is repeated during each PCR amplification cycle.

The data analysis module is configured to gather optical data from the optical detection module to both qualitatively determine the presence of one or more target oligonucleotide sequences in the ample and/or quantitatively determine the rate of PCR amplification (i.e., the number of amplicons produced) for the one or more target oligonucleotide sequences. A target sequence profile is generated by the data analysis module and the data is either transmitted to a remote server or stored on a local disk for comparison with a library of known sequence profiles to determine if the sample has been provided from an authentic article or not.

### Example 4

An Integrated Microsystem and Method for Rapid in-Field Detection of a Distinctive Marker

The integrated microsystem described in example 3 may be modified to eliminate thermal cycling by employing isothermal amplification. Therefore, the temperature control module may be omitted, and the rate of image capture may be increased to accommodate the faster cycling time for isothermal amplification.

The disclosures of each of the references, patents and published patent applications disclosed herein are each hereby incorporated by reference herein in their entireties.

In the event of a conflict between a definition herein and a definition incorporated by reference, the definition provided herein is intended.

Having described exemplary embodiments of the present invention, it is further noted that it is readily apparent to those of ordinary skill in the art that various modifications may be made without departing from the spirit and scope of the present invention.

What is claimed is:

1. A method of identifying articles in commerce, comprising:
tagging a raw material during said raw material's manufacturing process with a unique molecular marker in a home country in which the raw material was produced, the marker identifying home country information;
transporting the tagged raw material from the home country to a foreign country;
processing the tagged raw material in the foreign country to generate a processed product;
transporting the processed product to the home country;
analyzing the processed product to determine the presence of the marker in the processed product; and
determining whether the raw material originated in the home country in response to the detection of the marker.

2. The method as defined in claim 1, wherein in response to the marker being detected, a first tariff condition and/or tax is determined and in response to the marker not being detected a second tariff and/or tax condition is determined.

3. The method as defined in claim 1, wherein the marker includes a nucleic acid including a known DNA sequence.

4. The method as defined in claim 3, wherein the marker further comprises an IR upconverting phosphor (UCP) or a UV florophore.

5. The method as defined in claim 4, wherein the presence of the marker in the processed product is determined by using a technique selected from the group consisting of PCR, RT-PCR, Q-PCR, capillary electrophoresis, hybridization probes, FISH, RPA, LAMP.

6. The method as defined in claim 2, wherein the marker includes information and the information is retrieved from the marker and is compared to information stored in a database.

7. The method as defined in claim 6, wherein the nucleic acid is activated to enhance binding between the nucleic acid and the raw material to be tagged.

8. The method as defined in claim 1, wherein the raw material includes cotton.

9. The method as defined in claim 8, wherein the marker is applied to the cotton during a ginning stage.

10. The method as defined in claim 1, wherein the interrogation of the processed product occurs upon entry of the processed goods into the home country.

11. The method as defined in claim 1, wherein the taggent is applied to the raw material during the processing thereof.

12. The method as defined in claim 1, wherein the analysis of the processed product is undertaken by an in-field detection device.

13. A method of tracking articles in international commerce, comprising:
transporting a raw material tagged with a nucleic acid marker from a home country in which the raw material was produced to a non-home country, wherein said marker is associated with home country information;
subjecting the raw material to a processes in the non-home country to generate a value added product;
transporting the value added product to the home country; and
subjecting the value added product to an interrogation by an in-field detection device to detect the presence or absence of the nucleic acid marker, wherein in response to the nucleic acid marker being detected, a first tariff and/or tax condition is determined and in response to the nucleic acid marker not being detected a second tariff and/or tax condition is determined.

14. The method as defined in 13, wherein the nucleic acid marker is compared to a known sequence stored in a database.

15. The method as defined in claim 13, wherein the infield detection device includes a portable DNA sequencing device.

16. The method as defined in claim 15, wherein a microfluidic chip, a fully integrated microdevice or a lab on a chip configuration.

17. A system of tracking articles in international commerce, comprising:
a taggent including a molecular marker applied to raw material, the taggent including information associated with a country of origin;
a detection device for detecting the taggent in processed goods containing the raw material after the raw material has been processed to obtain country or origin information; and
an analysis device operably connected to the detection device for analyzing the detected taggent.

18. The system as defined in claim 17, wherein the marker includes a nucleic acid including a DNA sequence.

19. The method as defined in claim 18, wherein the marker further comprises an IR upconverting phosphor (UCP) or a UV florophore.

20. The system as defined in claim 18, wherein the marker comprises alkaline activated DNA, the alkaline activated DNA being produced by exposing the DNA to a solution of an alkali metal hydroxide having a concentration from about 0.001 M to about 1.0 M.

21. The system as defined in claim 20, wherein the detection device includes a portable scanner.

22. The system as defined in claim 18, wherein the detection device includes a PCR device.

23. The system as defined in claim 18, wherein the detection device includes a isothermal amplification device.

24. The system as defined in claim 18, wherein the detection device compares a property of the detected taggent with information stored in a database to determine the country of origin.

25. The system as defined in claim 19 wherein the detection device includes an in-field quantitative DNA amplification device.
